(19) United States
(12) Patent Application Publication
(10) Pub. No.: US 2021/0193286 A1
(43) Pub. Date: Jun. 24, 2021

(54) THERAPY-BASED DATABASE MODEL FOR GENERATING DRUG LIBRARIES

(71) Applicant: Fresenius Vial SAS, Brézins (FR)

(72) Inventor: Witold Moskal, Park Ridge, IL (US)

(21) Appl. No.: 17/055,447

(22) PCT Filed: Mar. 22, 2019
(86) PCT No.: PCT/EP2019/057225

§ 371 Date: Nov. 13, 2020

Related U.S. Application Data
(60) Provisional application No. 62/671,424, filed on May 15, 2018.

(51) Int. Cl. G16H 20/17; G06F 16/28; G16H 20/13; G16H 70/40; G16H 40/67; A61M 5/172

(52) U.S. Cl. CPC G16H 20/17; G06F 16/288; G16H 20/13; A61M 2205/52; G16H 40/67; A61M 5/172; G16H 70/40

(57) ABSTRACT

A system and method of generating a database based on an entity relationship model includes receiving a drug identifier from a user and storing the drug identifier in a drug entity in the database. The embodiments further include receiving drug delivery therapies from a user for a plurality of different therapies, storing each drug delivery therapy for the drug identifier in a therapy entity in the database, the drug entity having a one-to-many relationship with the therapy entity in the database, and generating a drug library based on the drug entity and the therapy entity. The drug library is transmitted to a medical device for use when programming the medical device to deliver a medicament.

### CROSS-REFERENCE TO RELATED APPLICATIONS

The present application claims the benefit of U.S. Provisional Patent Application No. 62/671,424, filed May 15, 2018, which application is incorporated by reference in its entirety. The present application is related to U.S. Provisional Patent Application No. 62/671,412 to Witold Moskal, titled “Drug Library Compiler for Patient Devices,” filed on May 14, 2018, which application is incorporated by reference in its entirety

### BACKGROUND

Infusion pumps are used to administer drugs and other medicaments to patients, typically in a clinical setting. An infusion pump provides a controlled amount of the medicament over time to the patient. The amount is administered pursuant to parameters entered by a clinician into the pump using a pump user interface.

Drug libraries are used on infusion pumps to provide further configuration beyond the software released by the manufacturer of the device and already operating on the device. Drug libraries can be user-configurable, for example by a pharmacist, and can include drug names, doses, limits to the upper and/lower ranges of administration parameters, and other operating configurations or parameters.

Some drug libraries use a drug-based model in which an entry in a drug library is indexed by drug name and concentration only.

### BRIEF DESCRIPTION OF THE DRAWINGS

FIG. 1 is a flow diagram illustrating an application context for the systems and methods described herein, according to an illustrative embodiment;

FIG. 2 is a flow diagram illustrating a medication administration workflow for the systems and methods described herein, according to an illustrative embodiment;

FIG. 3 is a flow diagram illustrating a drug library editor workflow for the systems and methods described herein, according to an illustrative embodiment;

FIG. 4 is an entity relationship diagram for the systems and methods described herein, according to an illustrative embodiment;

FIG. 5 is a flowchart of a method of generating a database based on an entity relationship model, according to an illustrative embodiment; and

FIG. 6 is a block diagram of a processing circuit, one or more components of which may be used in the computers or other processing components described herein.

### DETAILED DESCRIPTION OF ILLUSTRATIVE EMBODIMENTS

In some embodiments, a therapy-based model can be used for drug library creation to allow for programming multiple library entries for a same drug.

In some embodiments, a therapy-based model can be used for drug library creation to avoid unclear drug names that might be used with a drug-based model only.

In some embodiments, drug name is separated from clinical protocols using a concept called a therapy, such that a single drug may have multiple sets of protocols available for delivery of the drug in the drug library.

In some embodiments, the need for programming multiple “versions” of the same drug with unclear descriptions is avoided.

In some embodiments, a one-to-one relationship between drug and clinical protocols is avoided.

In one embodiment, a method of generating a database based on an entity relationship model comprises receiving a drug identifier from a user, storing the drug identifier in a drug entity in the database, receiving drug delivery therapies from a user for a plurality of different therapies, storing each drug delivery therapy for the drug identifier in a therapy entity in the database, the drug entity having a one-to-many relationship with the therapy entity in the database, generating a drug library based on the drug entity and the therapy entity, and transmitting the drug library to a medical device for use when programming the medical device to deliver a medicament. The database may comprise a drug library created using a drug library application that enables a pharmacist to create and/or distribute created drug libraries to supported infusion pumps. The medical device may be configured to provide medical functions or services to a patient. The medical device may provide the services by way of an invasive procedure, such as by using a needle in a procedure. The medical device may comprise an infusion pump.

The drug library may comprise a component of a Dose Error Reduction System or DERS. The DERS may alert a clinician of potential over- or under-delivery of a fluid by checking programmed doses against preset limits within the drug library. The limits may be soft limits or hard limits.

In some embodiments, once changes are made to a drug library using a drug library application, a server computer may be used to release or distribute the drug library to program infusion pumps.

In some embodiments, a drug library may comprise a list of drug entities organized by subcategories. The subcategories may comprise clinical locations, such as care areas. The subcategories may be associated with physical locations in the hospital. The subcategories may be named according to therapy type, such as epidural therapy. In some embodiments, a drug entity comprises a drug name and a concentration. A drug entity may comprise default values for concentration, dosing unit, and/or dosing limits. In some embodiments, a drug entity comprises only a drug name and/or concentration and does not include delivery parameters or therapies.

In some embodiments, a pump with DERS functionality may include a data log file that records data for programmed doses that triggered dose limit warnings.

In some embodiments, a drug library storage and distribution system may comprise an electronic processing circuit and memory configured to generate and store a drug library database. The drug library database may comprise a drug entity specifying a drug name and a drug concentration, a therapy entity specifying a clinical protocol for delivery of a drug, the drug entity having a one-to-plurality relationship with the therapy entity, the drug entity and the therapy entity both being included in a drug library entity. The processing circuit may be configured to distribute a drug library from the drug library entity to an infusion pump.

In some embodiments, a computer operating a drug library editor program may be used to facilitate the creation of a drug library by input from a pharmacist on an input device (e.g., keyboard, touch screen, data upload, etc.). The computer may release the drug library to medical devices. In some embodiments, the computer may be configured to receive an approval input from a competent authority via an input device before releasing the drug library to medical devices.

In some embodiments, each drug library may comprise definitions of drugs and therapies. A drug library application may be configured to provide a “release drug library” functionality. The release drug library functionality may be a programmed functionality which enables a pharmacist to organize drug libraries based on selected device configurations into profiles. The functionality may provide an option for clinical evaluation and approval by a clinician prior to distribution to a pump.

In some embodiments, a distribute drug library may be provided which enables a person to distribute selected drug libraries to individual pumps or groups of pumps over one or more computer networks.

In some embodiments, a database may comprise a drug library having an entity relationship model. The model may be a data structure that defines a drug to be infused by name and/or concentration. In some embodiments, the drug to be infused is defined without reference to a delivery protocol. In some embodiments, the drug name or identified may be separated from the clinical protocols (e.g., continuous rate protocol) into a therapy. A single drug may have multiple sets of protocols available for delivery, as stored in a relationship within the drug library.

In some embodiments, a drug library may comprise a drug entity data structure. The drug entity data structure may comprise a drug name field and optionally a concentration of the drug. In some embodiments, the drug entity data structure does not include delivery parameter or protocol data fields. The drug library may define a therapy data structure comprising a therapy name and defining one or more delivery parameters, such as protocol, rate, bolus, ramp rate, loading dose, etc.

In some embodiments, a “master” list of drugs is stored, each drug with corresponding therapies (linked by way of one or more pointers or other data constructs pointing in one-to-many fashion to therapies in a therapy entity) from which individual drug libraries for a specific clinical area can be built. In some embodiments, more than one therapy can be defined for a single drug within a single drug library.

In some embodiments, a master drug list entity may comprise links to drugs in a drug entity, thereby having a one-to-plurality relationship with a drug entity.

In some embodiments, all programming modes of a therapy or therapy entity may be delivered over a single channel and may be programmed within a single therapy. In some embodiments, a drug library editing and distribution system may comprise a computer memory and an electronic processing circuit. The memory may be configured to store a database of drug identifiers and therapy drug library in a first format, the drug library comprising drug data to be used by software on the patient devices to program the patient devices to administer a drug to a patient according to a protocol determined at least in part by a user. The processing circuit may be configured to receive a drug name and concentration programmed by a user, receive a name of a therapy programmed by a user, receive a clinical protocol programmed by a user, generate a drug record based on the drug name and concentration, generate a therapy record separate from the drug record based on the name of the therapy and the clinical protocol. In some embodiments, the therapy record may be associated with the drug record. In some embodiments, the drug record may be configured to be associated with a plurality of therapy records, each therapy record having a therapy name and a clinical protocol. In some embodiments, the processing circuit may be configured to generate a drug library file comprising the drug record and the therapy record and to transmit the drug library for distribution to one or more infusion pumps.

In some embodiments, drug entries and therapy entries may be separately created and/or managed. In some embodiments, a processing circuit may be configured to receive a drug identifier from a user and store the drug identifier as a drug entity in a database. In some embodiments, the processing circuit may be configured to receive drug delivery therapies from a user for a plurality of different therapies and store each drug delivery therapy for the drug identifier as a therapy entity in the database. In some embodiments, a drug entity may have a one-to-many relationship with a therapy entity in the database.

In some embodiments, any of a number of profiles may be created. A profile may correspond to a care area or use within a healthcare facility of a particular medical device. In some embodiments, profiles may be associated with drug libraries. In some embodiments, a data set or drug library may be created. The data set or drug library may comprise one or more profiles to be associated with a pump or group of pumps or other medical devices.

In some embodiments, creating a drug for a drug entity may comprise a user specifying a drug name, drug concentration, drug category (e.g., analgesic, antiviral, etc.), etc. In some embodiments, creating a therapy can comprise first selecting a drug name and then selecting therapy protocol, parameters and/or configurations. Parameters may comprise a dose unit, whether the protocol is fixed or user-programmable within a range of values, a default programming value, a drug concentration, a textual message to be displayed on the medical device, whether a drug is configurable in flow rate or dose rate (or flow rate only), and/or other parameters.

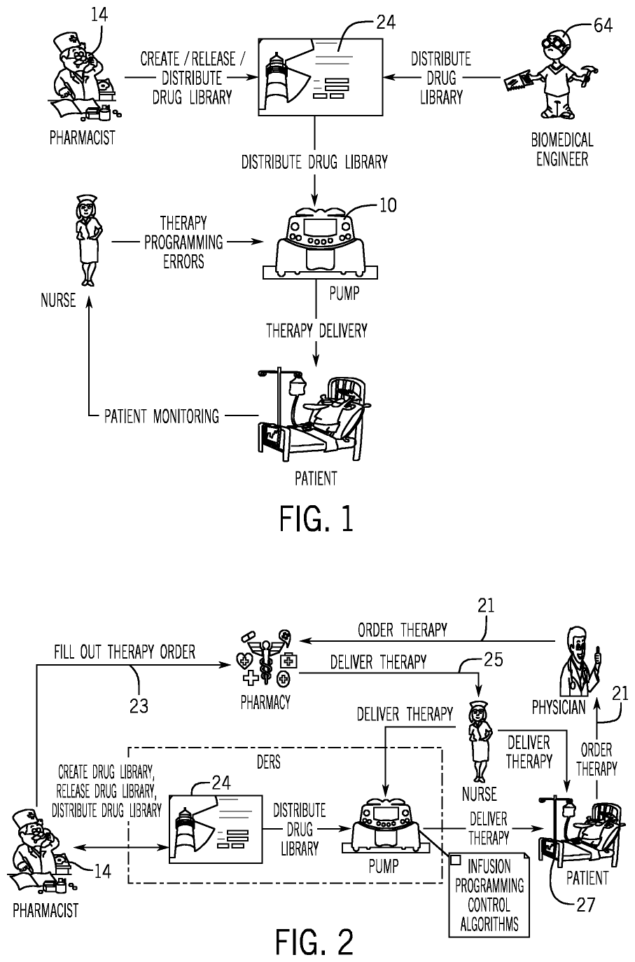

With reference to FIG. 1, a Dose Error Reduction System (DERS) may comprise at least two components. A first component of a DERS is an infusion pump 10 which may comprise control algorithms implemented in the pump to prevent errors in dose programming. DERS allows infusion pumps to warn users of incorrect medication orders, calculation errors, or incorrect infusion programming that would result in under-or overdose of a drug. DERS may provide a defense mechanism against medication programming errors. A second component of DERS is a drug library application 24 that enables pharmacist 14 to create and distribute created drug libraries to supported infusion pumps 10.

DERS may be applied to a variety of medical devices or patient devices, such as a device configured to provide medical functions or services to a patient (including a blood or organ donor) in any clinical, hospital, home care, or other setting, by way of invasive procedure (e.g., using a needle in a procedure) or otherwise. Infusion pump 10 may be any of a variety of infusion pumps, such as a large volume or general purpose infusion pump (i.e. one configured to dispense from a bag instead of a syringe), a patient-controlled analgesia (PCA) pump, an elastomeric pump, a syringe pump, an enteral or parenteral feeding pump, an insulin pump, an ambulatory pump, etc. Infusion pumps that have a DERS may be referred to as “smart” pumps.

A DERS may alert clinicians (e.g., nurses, physicians, etc.) of potential over- or under-delivery of fluid by checking programmed doses (programmed by an end user at the pump) against preset limits (within a drug library or data set) which may be specific to a drug and/or specific to a clinical application or location or care area (e.g., epidural, neonatal intensive care unit (NICU), medical/surgical unit, etc.). If the programmed dose is outside the limits, the pump alerts a clinician and can either require confirmation before beginning delivery (soft limit) or not allow delivery at all (hard limit).

To program, create, or edit a drug library, a user 64 (e.g., a pharmacist, biomedical engineer, etc.) logs into a server computer or terminal (e.g., pharmacy computer located or disposed in a pharmacy to be programmed by a pharmacist) in communication with a server computer. The user creates, edits, and/or selects one or more datasets to be programmed into or downloaded to infusion pump(s) 10 to satisfy needs of an end user workflow in the deployment environment (e.g., hospitals, health care facilities, clinics, etc.). The dataset may comprise data that the infusion pump uses in its operation. For example, the dataset may comprise drug name and/or concentration, as well as drug programming parameters that provide default values and/or limits to a user's ability to program the infusion pump. For example, a data set may comprise hard limits and/or soft limits to different pump programming parameters, such as infusion rate, dose, infusion time or duration, etc. The limits of the data set may be different for different drugs, and may include a “drug X” data set for a drug not known by the data library. Once changes are made to the data set or library, a server computer (e.g., a drug library distribution server, a release server, or other server computer) may be used to release and distribute data sets to configure, update and/or otherwise program infusion pumps 10, which may be done individually, by care area, universally, etc., with the new data set created by the pharmacist. The user may select a date and time after which the infusion pump will receive the new dataset. The patient devices may be clinician-programmable infusion pumps.

Clinical staff, typically nurses, pharmacists, and/or physicians, collaborate to develop custom drug libraries to match a facility's particular care practices. A library may be created on a dedicated application 24 and then distributed to each pump, and may be updated regularly as new drugs or new uses for existing drugs emerge. Drug libraries may be revised every few months or so to add new drugs and to shift dosing limits to better fit clinical practice.

In some embodiments, a drug library is based on the facility's dosing protocols and comprise a list of drug entities organized by subcategories that are identified by the facility. These subcategories may be referred to informally as “clinical locations,” or they can be designated however the facility chooses. While the subcategories often match care areas (e.g., NICU, medical/surgical, etc.), they can also be named according to physical locations in the hospital (e.g., 5 West) or according to the therapy type (e.g., epidural). Each drug entity comprises a drug name and optionally a concentration, and further may include associated default values or parameters for concentration, dosing unit, dosing limits, etc. When a user selects a drug entity from the library, the pump uses the associated defaults along with additional user input to program the infusion. In this way, well-designed libraries help control dosing errors by reducing the need for manual input and calculations. In some embodiments, a drug entity comprises only a drug name and/or concentration and does not include delivery parameters or therapies.

A nurse, biomedical engineering staff, or other user may, upon seeing a notification at pump 10 that a new drug library has been downloaded, cycle the power on the pump to install or activate the new dataset. Pump 10 may be configured to confirm via a notification that a dataset is available for upgrading or updating the pump. Once the dataset is upgraded, pump 10 may notify a server computer of upgrade status (e.g., upgrade complete or successful, upgrade failed or error, etc.). Pump 10 may then disconnect from communications with the server computer for security purposes. After cycling the power, the nurse is able to infuse under control of the new dataset downloaded.

In some embodiments, pumps with DERS functionality may also include a log that records data for programmed doses that trigger dose limit warnings. Clinicians may later use log-analysis software to aggregate the alert information from multiple pumps' logs to find opportunities to improve clinical practice and decide whether they need to revise the drug library. The data garnered from such log analysis can be useful for performing proactive risk assessments of higher-risk processes. Furthermore, log analysis allows facilities to check the results of changes that have been implemented.

The use of DERS may improve, but need not change, the existing infusion management workflow of a healthcare facility by reducing programming errors when DERS is used in the infusion management process. Referring to FIG. 2, a medication administration workflow will be described that may deploy the systems and methods described herein. At line 21, a physician orders a therapy from a pharmacy within the facility. Pharmacist 14 may fill out the therapy order (line 23) and the pharmacy may deliver the therapy via a clinician (line 25) to a patient 27 using pump 10. A computer operating a drug library editor or application program 24 may be used to facilitate the creation of the drug library by pharmacist 14, the release of the drug library (which may comprise obtaining an approval from a competent authority), and/or the distribution of the drug library.

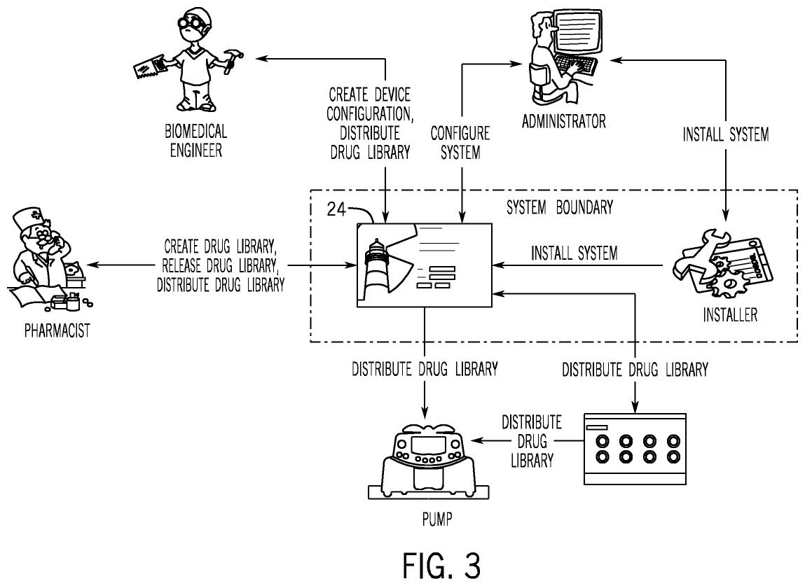

Referring now to FIG. 3, drug library application 24 as a component of DERS may be used to: (1) create; (2) release; and/or (3) distribute drug libraries to supported pumps. In addition, pump installation and pump configuration functions may be provided to simplify deployment and configuration of the system prior to its first intended use. Drug library application 24 may be configured to provide a “create drug library” functionality which enables a pharmacist to create, modify and/or delete drug libraries with their definitions of drugs and therapies, as well as other parameters, such as patient weight for example. Drug library application 24 may be configured to provide a “release drug library” functionality which enables a pharmacist to organize drug libraries with selected device configurations into profiles, and to release complete profiles for clinical evaluation and approval, and subsequent distribution to the pump. Drug library application 24 may be configured to provide a “distribute drug library” functionality, which enables a biomedical engineer to distribute a released drug library to selected pump(s). The biomedical engineer may distribute selected drug libraries to an individual pump via direct connection with serial cable (line 27), or to multiple pumps via a distribution mechanism provided by server computer (line 29).

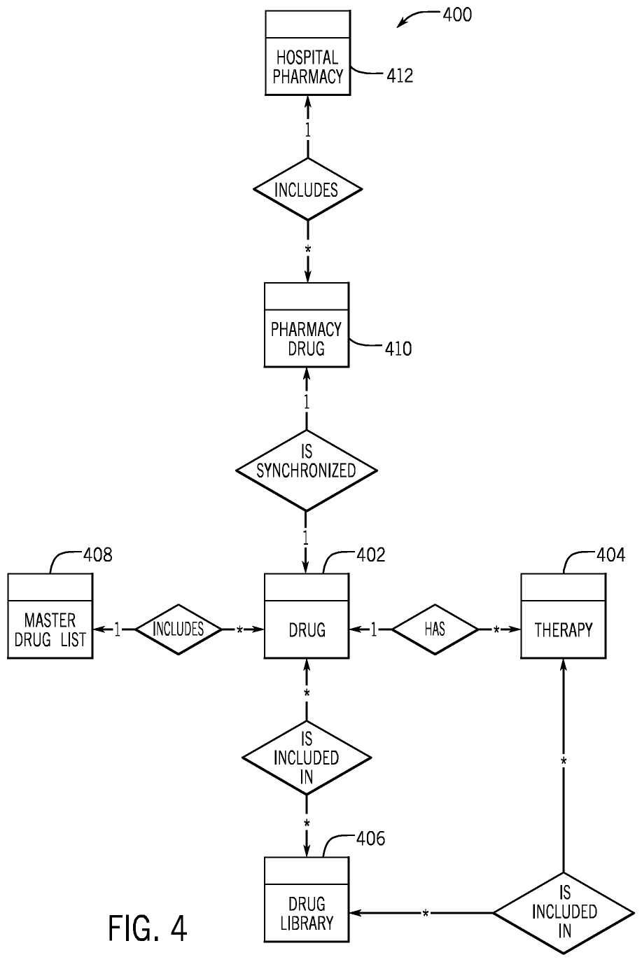

Referring now to FIG. 4, systems and methods for generating a database based on an entity relationship model will be described. In some embodiments, the database uses a model that represents a drug to be in infused (e.g., amoxicillin, etc.) by name and/or concentration and, in some embodiments, without a delivery protocol. Some drug-based data models suffer from the fact that the definition or name of the drug corresponds in a one-to-one relationship with a defined clinical protocol, and therefore such models prevent the same drug to be used with a different set of clinical protocols. For example, in typical drug library model, when a drug is defined with a name Amoxicillin and a continuous rate protocol, there can only be one way to administer Amoxicillin (namely with a continuous rate protocol), unless “Amoxicillin1” or “AmoxicillinOne” is created with a different clinical protocol. This renaming can lead to errors in programming if the drug name differentiation is not clear to all users.

In some embodiments, a therapy-based drug library model may be implemented. In some embodiments, the drug name or identifier may be separated from the clinical protocols into a concept called therapy, such that a single drug, e.g. Amoxicillin, may have multiple sets of protocols available for delivery of the drug, without the need for creating another “version” of the drug.

Referring again to FIG. 4, an entity-relationship diagram is shown illustrating a therapy-based drug library model, according to an illustrative embodiment. An entity-relationship model is used to define a data or information structure which can be implemented in a database, such as a relational database. Each entity in the database may comprise a plurality of records. For example, drug entity 402 may comprise a plurality of drug records, files or entries within a table of a database. Therapy entity 404 may comprise a plurality of therapy records, files or entries, each therapy data element comprising a therapy name and defining one or more delivery parameters. While an embodiment is described with reference to an entity-relationship model, other models or techniques may be used to implement the principles and teachings provided herein. In some embodiments, this model may have the benefit of allowing synchronization of the list of drugs with the hospital pharmacy system without regard for specificity of the infusion clinical protocols. As indicated, drug entity 402 is synchronized with pharmacy drug entity 410. In this case, drug entity 402 comprises drug name and optionally concentration, but not delivery parameters or protocol. In some embodiments, a “master” list of drugs is created with their corresponding therapies (linked by way of a pointer or other data construct pointing in one-to-many fashion to therapies in therapy entity 404) from which individual drug libraries for a specific clinical area can be built. In some embodiments, more than one therapy can be defined for a single drug within a single drug library.

A drug library storage and distribution system 400 may comprise a processing circuit or other computing resource and a memory configured to generate and store a drug library database. The drug library database may comprise a drug entity 402 specifying a drug name and/or a drug concentration. The drug library database may comprise a therapy entity 404 specifying a clinical protocol for delivery of a drug. The clinical protocol may comprise one or more parameters, such as a bolus, a continuous rate, a ramp rate, a loading dose, and/or other protocols.

As shown, drug entity 402 may have a one-to-plurality or one-to-many relationship with therapy entity 404. A drug library entity 406 may be generated pursuant to a user's specifications, which may include data from the drug entity and/or from the therapy entity. The drug library may then be released, distributed, etc. to select patient devices.

In some embodiments, a master drug list entity 408 is provided which includes or comprises or links to drugs in drug entity 402, thereby having a one-to-plurality relationship with drug entity 402.

In some embodiments, the drug library database may further comprise a pharmacy drug entity 410 which is synchronized with drug entity 402. Pharmacy drug entity 410 specifies data stored in a separate computer (hospital pharmacy computer entity 412) associated with a hospital pharmacy, whereby synchronization of the pharmacy drug entity 410 with the drug entity 402 does not modify the therapy entity 404. Drug library 406 may comprise one or more therapies for the drug name and drug concentration specified in the drug entity, allowing a user of pump 10 to select a therapy for the drug and/or select the drug by drug name, thereby pulling up parameters for the drug name.

In some embodiments, therapy entity 404 may specify a single therapy comprising a combination of different clinical protocols, for example, protocols to be applied in sequence, at different times, etc.

In some embodiments, the model may have a drug-to-therapy one-to-many relationship. In some embodiments, a therapy does not refer to different pump types (e.g., such as a PCA pump, etc.), but rather comprises a definition of a delivery mode of a drug, comprising such protocols as a continuous rate delivery and/or other parameters. In some embodiments, the model of FIG. 4 may be implemented as an algorithm embodied on a tangible medium. The algorithm may embody various means for performing the steps illustrated in the model and or otherwise described herein. In some embodiments, a therapy entity may comprise a combination of different programming modes, such as loading dose, bolus, continuous rate, etc. associated with a single therapy or therapy data file.

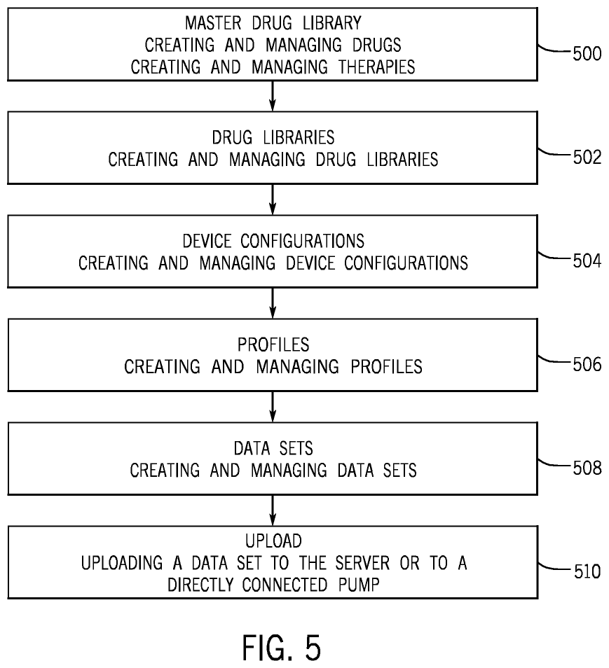

In some embodiments, all programming modes of a therapy or therapy entity may be delivered over a single channel and may be programmed within a single therapy. Referring now to FIG. 5, a method of generating a database based on an entity relationship model will be described. At a block 500, drug entries for drug entity 402 may be created and/or managed. Therapy entries for therapy entity 404 may also be created and/or managed. For example, a processing circuit may be configured to receive a drug identifier from a user and store the drug identifier in drug entity 402 in the database. Further, a processing circuit may be configured to receive drug delivery therapies from a user for a plurality of different therapies and store each drug delivery therapy for the drug identifier in therapy entity 404 in the database. As mentioned, drug entity 402 has a one-to-many relationship with therapy entity 404 in the database.

At a block 502, the processing circuit may be configured to generate a drug library based on the drug entity and the therapy entity by allowing a user to select data from the entities for compilation in a drug library to be distributed to pumps, for example pursuant to a distribution policy.

At a block 504, device configurations may be created and/or managed, such as a total air volume allowed over a period of time before an air-in-line alarm is triggered, a silence key duration, a keep-vein open (KVO) flow rate, or any of a number of other device configurations.

At a block 506, any of a number of profiles may be created and/or managed. For example, a user may enter names of operational profiles, which may correspond to care areas or uses within a healthcare facility for the particular pumps having the profiles. Profiles may further be associated with drug libraries from blocks 500, 502.

At a block 508, a data set may be created and/or managed. A data set may comprise one or more validated profiles to be associated with a pump, each profile further comprising one or more data libraries.

At a block 410, the processing circuit may be configured to transmit the drug library (as part of data set, profile, or otherwise) to a medical device for use when programming the medical device to deliver a medicament.

In some embodiments, creating a drug for drug entity 402 may comprise specifying one or more of a drug name, drug concentration, a drug category for the drug (e.g., analgesic, antiviral, homecare, hospital, vasopressor, etc.), etc. Drug parameters may be entered as well (e.g., bolus, max/min limits, default delivery rate, etc.), or, in alternative embodiments, drug entity 402 may comprise merely drug name, drug concentration and/or drug category, with a later-defined therapy to incorporate other delivery parameters.

In some embodiments, creating a therapy may comprise first selecting a drug name previously created and optionally stored in drug entity 402 and then selecting one or more therapy protocols, parameters, or configurations, such as a dose unit, whether the protocol is fixed or within a range (the latter allowing minimum, default and maximum values), a drug concentration, clinical messages such as advisories, whether a drug is configurable in flow rate or dose rate (or flow rate only), whether volume/time infusion is allowed, whether volume/rate infusion is allowed, whether time/rate infusion is allowed, whether the therapy will include a ramp mode (defined by total volume, total infusion time, ramp-up and ramp-down times and/or plateau flow rate), continuous flow rate settings, dose or volume over time settings, volume to be infused settings, loading dose settings, programmed bolus settings, direct bolus settings, or any other parameters comprising a delivery protocol or set of constraints on the pump. A clinical protocol may be a protocol for delivering a drug or other medicament (or feeding formulation) comprising one or more of the parameters described above.

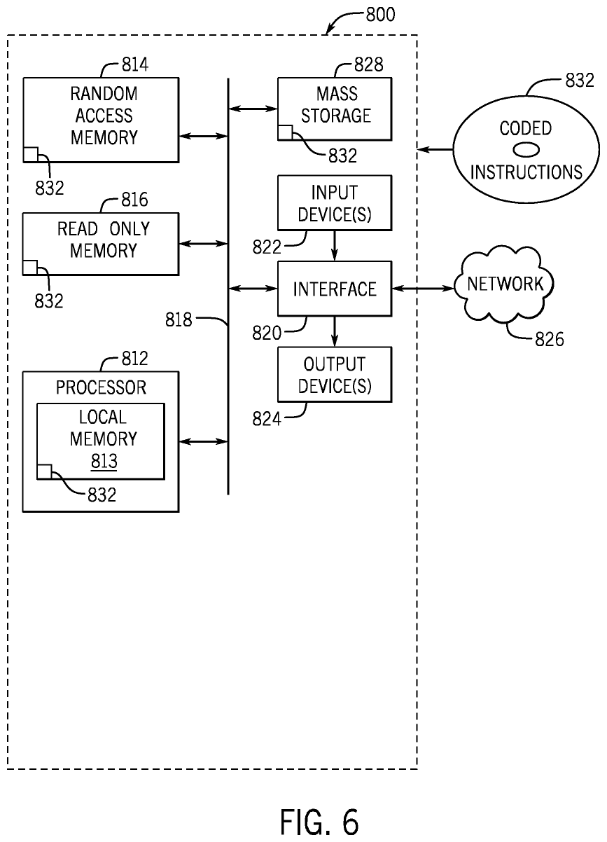

FIG. 6 is a block diagram of processing circuit components, one or more of which may be used in the computing devices described herein (e.g., server computer, pharmacy computer, patient device, uploader computer, etc.). In alternate embodiments, the systems and methods described herein may be implemented on a single server computer, a plurality of server computers, a server farm, a cloud server environment, or using other computer resources. Servers and patient device 10 may comprise analog and/or digital circuit components forming processing circuits configured to perform the steps described herein. The processing circuits may comprise discrete circuit elements and/or programmed integrated circuits, such as one or more microprocessors, microcontrollers, analog-to-digital converters, application-specific integrated circuits (ASICs), programmable logic, printed circuit boards, and/or other circuit components. Servers and patient device 10 may each comprise a network interface circuit configured to provide communications over one or more networks with each other and/or with other devices. Network interface circuits among devices may comprise digital and/or analog circuit components configured to perform network communications functions. The networks may comprise one or more of a wide variety of networks, such as wired or wireless networks, wide-area, local-area or personal-area networks, proprietary or standards-based networks, etc. The networks may comprise networks such as an Ethernet network, networks operated according to Bluetooth protocols, IEEE 802.11x protocols, cellular (TDMA, CDMA, GSM) networks, or other network protocols. The network interface circuits may be configured for communication of one or more of these networks and may be implemented in one or more different sub-circuits, such as network communication cards, internal or external communication modules, etc.

FIG. 6 is a block diagram of an example processor platform 800 capable of executing the instructions described herein and/or to implement the example systems described herein. The processor platform 800 can be, for example, a server, a personal computer, a mobile device (e.g., a cell phone, a smart phone, a tablet such as an iPad™), a personal digital assistant (PDA), an Internet appliance, or any other type of computing device.

The processor platform 800 of the illustrated example includes a processor 812. The processor 812 of the illustrated example is hardware. For example, the processor 812 can be implemented by one or more integrated circuits, logic circuits, microprocessors or controllers from any desired family or manufacturer. In the illustrated example, the processor 812 is structured to be programmed with components to implement the functions described herein, such as those applying criteria to software version indicators.

The processor 812 of the illustrated example includes a local memory 813 (e.g., a cache). The processor 812 of the illustrated example is in communication with a main memory including a volatile memory 814 and a non-volatile memory 816 via a bus 818. The volatile memory 814 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 816 may be implemented by flash memory and/or any other desired type of memory device. Access to the main memory 814, 816 is controlled by a memory controller.

The processor platform 800 of the illustrated example also includes an interface circuit 820. The interface circuit 820 may be implemented by any type of interface standard, such as those described hereinabove.

In the illustrated example, one or more input devices 822 are connected to the interface circuit 820. The input device(s) 822 permit(s) a user to enter data and commands into the processor 812. The input device(s) can be implemented by, for example, an audio sensor, a microphone, a camera (still or video), a keyboard, a button, a mouse, a touchscreen, a track-pad, a trackball and/or a voice recognition system.

One or more output devices 824 are also connected to the interface circuit 820 of the illustrated example. The output devices 824 can be implemented, for example, by display devices (e.g., a light emitting diode (LED), an organic light emitting diode (OLED), a liquid crystal display, a cathode ray tube display (CRT), a touchscreen, a tactile output device, a printer and/or speakers). The interface circuit 820 of the illustrated example, thus, typically includes a graphics driver card, a graphics driver chip or a graphics driver processor.

The interface circuit 820 of the illustrated example also includes a communication device such as a transmitter, a receiver, a transceiver, a modem and/or network interface card to facilitate exchange of data with external machines (e.g., computing devices of any kind) via a network 826 (e.g., an Ethernet connection, a digital subscriber line (DSL), a telephone line, coaxial cable, a cellular telephone system, etc.).

The processor platform 800 of the illustrated example also includes one or more mass storage devices 828 for storing software and/or data. Examples of such mass storage devices 828 include floppy disk drives, hard drive disks, compact disk drives, Blu-ray disk drives, RAID systems, and digital versatile disk (DVD) drives.

Coded instructions 832 representing the flow diagram of FIG. 5 or other steps described herein may be stored in the mass storage device 828, in the volatile memory 814, in the non-volatile memory 816, and/or on a removable tangible computer readable storage medium such as a CD or DVD.

Certain embodiments contemplate methods, systems and computer program products on any tangible machine-readable media to implement functionality described above. Certain embodiments can be implemented using an existing computer processor, or by a special purpose computer processor incorporated for this or another purpose or by a hardwired and/or firmware system, for example.

Some or all of the system, apparatus, and/or article of manufacture components described above, or parts thereof, can be implemented using instructions, code, and/or other software and/or firmware, etc. stored on a tangible machine accessible or readable medium and executable by, for example, a processor system. Tangible computer readable media include a memory, DVD, CD, etc. storing the software and/or firmware, but do not include a propagating signal.

Additionally or alternatively, the example processes described herein may be implemented using coded instructions (e.g., computer readable instructions) stored on a non-transitory computer readable medium such as a hard disk drive, a flash memory, a read-only memory, a compact disk, a digital versatile disk, a cache, a random-access memory and/or any other storage media in which information is stored for any duration (e.g., for extended time periods, permanently, brief instances, for temporarily buffering, and/or for caching of the information).

Certain embodiments described herein can omit one or more of the method steps and/or perform the steps in a different order than the order listed. For example, some steps cannot be performed in certain embodiments. As a further example, certain steps can be performed in a different temporal order, including simultaneously, than listed above. While the exemplary embodiments have been described with reference to an infusion pump, the teachings herein may be applied to other medical devices, such as apheresis devices (e.g., plasmapheresis, blood therapy, etc.) or other devices that are invasive or noninvasive, that interface with a human patient via a needle in the patient's skin, insulin pumps (e.g., internal or external to the body cavity), medical imaging devices (e.g., CT scanners, x-ray imagers, magnetic resonance imaging). The teachings may also be applied outside the medical field to any computing devices, such as mobile phones, tablet computers or other computers configured to be operated while held in a human hand, laptops, personal computers, and other networked computers.

Certain examples facilitate management of medical devices including blood collection or apheresis devices, infusion pumps, drug delivery pumps, and/or other medical devices. For example, an infusion pump infuses fluids, medication, or nutrients into a patient. An infusion pump can be used intravenously, subcutaneously, arterially, and/or epidurally, for example. For example, an infusion pump can administer injections at a variety of rates (e.g., injections too small for an intravenous (IV) drip (e.g., 0.1 mL per hour), injections per minute, injections with repeated boluses, patient-controlled injections up to maximum number per hour, or injections of fluids whose volumes vary by time of day, etc.).

In certain examples, an operator (e.g., a technician, nurse, etc.) provides input to patient devices regarding type of infusion, mode, and/or other device parameter, with the protocol defined by a drug library comprising a therapy. For example, continuous infusion provides small pulses of infusion (e.g., between 500 nanoliters and 10 milliliters), with a pulse rate based on a programmed infusion speed. Intermittent infusion alternates between a high infusion rate and a low infusion rate with timing programmable to keep a cannula open, for example. Patient-controlled infusion provides on-demand infusion with a preprogrammed ceiling to avoid patient intoxication. The infusion rate is controlled by a pressure pad or button that can be activated by the patient, for example. Infusion pumps can include large volume pumps (e.g., for nutrient solution delivery to feed a patient), small-volume pumps (e.g., for medicine delivery), etc.

Certain examples determine and/or update a data set distribution policy associated with a medical device data management system. If a data set distribution policy has been created, then a new or updated data set (e.g., a new or updated drug library) can be distributed to one or more medical devices, even if one or more of the target medical devices are currently operating (e.g., pump(s) are currently infusing drug into a patient). Thus, data set distribution does not impact the pump's activity.

In certain examples, a data set defines a drug library and/or instruction set for a medical device such as a “smart” infusion pump, apheresis device, etc. For example, “smart” infusion pumps utilize a drug library or other dose error reduction software to perform functions that assist healthcare providers with programming and calculating drug dose and delivery rates. The drug library is a database or data set that stores drug dosing information, including dosing limits, concentration, infusion parameters, and drug specific advisories, for example. Drug library instructions can help reduce or prevent medication errors and associated patient harm, for example.

Distribution of a data set to medical devices can occur directly at the device and/or remotely over a network (e.g., from a data management system to multiple medical devices, etc.).

In certain examples, a pharmacy controls the data set and distribution, and the end user (e.g., nurse, technician, etc.) is not given decision-making authority but must accept the data set to continue using the device. The device management system sends out a data set, and a receiving device is configured to activate the new data set on next reboot. The downloaded data set is held in a download buffer, for example, and, once the device is powered on, the device programs the data set into active memory. The user is then informed of the new data set and instructed to use the device or turn the device off but cannot revert to a prior version of the data set.

While the embodiments have been described with reference to certain details, it will be understood by those skilled in the art that various changes can be made and equivalents can be substituted without departing from the scope described herein. In addition, many modifications can be made to adapt a particular situation or material to the teachings without departing from its scope. Therefore, it is intended that the teachings herein not be limited to the particular embodiments disclosed, but rather include additional embodiments falling within the scope of the appended claims.

1. A method of generating a database based on an entity relationship model, comprising:
receiving a drug identifier from a user;
storing the drug identifier in a drug entity in the database;
receiving drug delivery therapies from a user for a plurality of different therapies;
storing each drug delivery therapy for the drug identifier in a therapy entity in the database, the drug entity having a one-to-many relationship with the therapy entity in the database;
generating a drug library based on the drug entity and the therapy entity; and
transmitting the drug library to a medical device for use when programming the medical device to deliver a medicament.

2. The method of claim 1, wherein each drug delivery therapy comprises at least one clinical protocol selected from a group comprising a bolus, a continuous rate, a ramp rate and a loading dose.

3. The method of claim 2, wherein the drug identifier comprises a drug name and a drug concentration.

4. The method of claim 3, wherein the drug entity comprises the drug name and does not include delivery parameters.

5. A drug library storage and distribution system, comprising:
a processing circuit and memory configured to generate and store a drug library database comprising:
a drug entity specifying a drug name and a drug concentration; and
a therapy entity specifying a clinical protocol for delivery of a drug,
the drug entity having a one-to-plurality relationship with the therapy entity, and
the drug entity and the therapy entity both being included in a drug library entity,
wherein the processing circuit is configured to distribute a drug library from the drug library entity to an infusion pump.

6. The drug library storage and distribution system of claim 5, the drug library database further comprising a master drug list entity having a one-to-plurality relationship with the drug entity.

7. The drug library storage and distribution system of claim 5, the drug library database further comprising a pharmacy drug entity being synchronized with the drug entity, the pharmacy drug entity specifying data stored in a separate computer associated with a hospital pharmacy, whereby synchronization of the pharmacy drug entity with the drug entity does not modify the therapy entity.

8. The drug library storage and distribution system of claim 5, wherein the drug library comprises a plurality of therapies for the drug name and drug concentration specified in the drug entity.

9. The drug library storage and distribution system of claim 5, wherein the clinical protocol of the therapy entity is selected from a group comprising a bolus, a continuous rate, a ramp rate and a loading dose.

10. The drug library storage and distribution system of claim 9, wherein the therapy entity may specify a single therapy comprising a combination of different clinical protocols.

11. A drug library editing and distribution system, comprising:
a memory configured to store a database of drug identifiers and therapy drug library in a first format, the drug library comprising drug data to be used by software on the patient devices to program the patient devices to administer a drug to a patient according to a protocol determined at least in part by a user; and
a processing circuit configured to:
receive a drug name and a drug concentration programmed by a user;
receive a name of a therapy programmed by a user;
receive a clinical protocol programmed by a user;
generate a drug record based on the drug name and the drug concentration;
generate a therapy record separate from the drug record based on the name of the therapy and the clinical protocol, the therapy record being associated with the drug record, wherein the drug record is configured to be associated with a plurality of therapy records, each therapy record having a therapy name and a clinical protocol;
generate a drug library file comprising the drug record and the therapy record; and
transmit the drug library for distribution to one or more infusion pumps.

12. The system of claim 11, further comprising an infusion pump comprising operating software, the infusion pump configured to receive the drug library and constrain operation of the operating software based at least in part on the drug library.

13. The system of claim 12, wherein the infusion pump is a clinician-programmable infusion pump.
